(19) United States
(12) Patent Application Publication
(10) Pub. No.: US 2022/0000754 A1
(43) Pub. Date: Jan. 6, 2022

(54) PERFUME GEL

(71) Applicant: L'OREAL, Paris (FR)

(72) Inventors: Véronique PERRAULT, Chevilly La Rue (FR); Christine NOEL, Chevilly La Rue (FR)

(21) Appl. No.: 17/292,209

(22) PCT Filed: Dec. 12, 2019
(86) PCT No.: PCT/EP2019/084963

§ 371 Date: May 7, 2021

(51) Int. Cl. A61K 8/81; A61K 8/04; A61Q 13/00; A61K 8/34

(52) U.S. Cl. CPC A61K 8/8158; A61K 8/042; A61K 8/34; A61K 8/8152; A61Q 13/00

(57) ABSTRACT

The present invention relates to a fragrancing, in particular cosmetic and/or dermatological, composition, comprising:

a physiologically acceptable aqueous medium comprising at least ethanol;
at least one sulfonic polymer chosen from homopolymers of acrylamido-2-methylpropanesulfonic acid or salts thereof and copolymers of acrylamido-2-methylpropanesulfonic acid or salts thereof and of one or more nonionic monomers;
at least one crosslinked copolymer of acrylic acid and of C₁₀-C₃₀ alkyl acrylate; and
at least 5% by weight of at least one fragrancing substance, relative to the total weight of the composition.

The present invention relates to a fragrancing composition in the form of an aqueous-alcoholic gel comprising at least 5% by weight of at least one fragrancing substance, relative to the total weight of composition.

Fragrancing compositions make it possible to fragrance the body by touch application. Among these compositions are in particular formulas in the form of an aqueous-alcoholic gel.

The formulations in the form of an aqueous-alcoholic gel currently available on the market generally comprise a large amount of alcohol (i.e. at least 30% by weight), to ensure their long-term preservation.

However, such formulas generally contain a small amount of fragrance (for example of the order of 1% by weight), which therefore fades rapidly over time.

There is thus a need for fragrancing compositions based on aqueous-alcoholic gel which contain a large amount of fragrance and which are stable and homogeneous.

The aim of the invention is to solve the abovementioned technical problems. In particular, one objective consists in providing a fragrancing cosmetic composition in the form of an aqueous-alcoholic gel, which is stable and homogeneous. Such a fragrancing composition contains a very high concentration of fragrance, and allows fragrancing by touch on the pulse points (i.e. areas of the skin located behind the ears, on the inside of the wrists, on the inside of the elbow), is pleasant on application (creamy application), and has a powdery finish after application to the skin (not rough, not sticky, and is soft and glides). The fragrancing composition also exhibits freshness on application.

The inventors have now discovered, surprisingly, that the combination of two specific gelling polymers, in an aqueous medium comprising ethanol and containing a very high concentration of fragrance, makes it possible to obtain aqueous-alcoholic gels that are stable (i.e. after a month at a temperature of 45° C.) and pleasant on application, with a fresh feel and a powdery finish. Surprisingly, the composition remains homogeneous (no phase separation observed).

A subject of the present invention is therefore a fragrancing, in particular cosmetic and/or dermatological, composition, comprising:

a physiologically acceptable aqueous medium comprising at least ethanol;
i) at least one sulfonic polymer chosen from homopolymers of acrylamido-2-methylpropanesulfonic acid or salts thereof and copolymers of acrylamido-2-methylpropanesulfonic acid or salts thereof and of one or more nonionic monomers;
ii) at least one crosslinked copolymer of acrylic acid and of C₁₀-C₃₀ alkyl acrylate; and
iii) optionally at least one organic thickening polymer other than i) and ii);
   at least 5% by weight of at least one fragrancing substance, relative to the total weight of the composition.

The present invention also relates to a method of cosmetic and/or esthetic care of, in particular for fragrancing, keratin materials, comprising the topical application to keratin materials, preferably to the skin, of a composition according to the invention.

The term “keratin materials” is intended to mean the skin and/or the lips and/or the hair.

The term “at least one” is intended to mean one or more.

Preferably, the composition according to the invention is in the form of an aqueous-alcoholic gel.

The term “aqueous-alcoholic gel” is intended to mean a gel comprising an aqueous medium comprising at least ethanol. The composition according to the invention is in particular in the form of a gel, and comprises specific “gelling polymers”. For the purposes of the present invention, the term “gelling polymer” is intended to mean a polymer which makes it possible to significantly increase the viscosity of the composition.

Viscosity

The compositions according to the invention preferably have a viscosity of between 9 and 40 Poises (0.9 to 4 Pa s), preferably between 26 and 35 Poises (2.6 to 3.5 Pa s), preferably between 26 and 30 Poises (2.6 to 3 Pa·s).

The viscosity measuring protocol is as follows:

The viscosity is measured with a Rheomat viscometer equipped with a spindle 2, 3 or 4.

The measurements are carried out at a temperature of 25° C.+/−0.5° C. after 10 minutes of rotation of the spindle at a speed of 200 revolutions/minute.

The constituents of the composition according to the invention are now described in greater detail.

Aqueous Phase

The composition of the invention comprises a physiologically acceptable aqueous medium comprising at least ethanol. The term “physiologically acceptable” is intended to mean a medium that is compatible with keratin materials.

The composition according to the invention comprises an aqueous medium comprising at least water. The aqueous medium also comprises at least ethanol.

The aqueous medium may comprise at least one other organic solvent which is soluble in water at 25° C., chosen for example from linear or branched C₃-C₄ alkanols, such as isopropanol, propanol or butanol; polyols having in particular from 2 to 20 carbon atoms, preferably from 2 to 6 carbon atoms, such as glycerol, diglycerol, propylene glycol, isoprene glycol, dipropylene glycol, butylene glycol, hexylene glycol, 1,3-propanediol, pentylene glycol, polyethylene glycols having from 2 to 200 ethylene oxide units; and mixtures thereof.

Preferably, the aqueous medium also comprises at least one polyol having from 2 to 20 carbon atoms, preferably from 2 to 6 carbon atoms.

The composition preferably comprises from 30% to 55% by weight of water relative to the total weight of the composition, preferably from 35% to 50%.

Preferably, the composition comprises from 15% to 40% by weight of ethanol relative to the total weight of the composition, preferably from 15% to 30% by weight, preferably from 15% to 25% by weight.

The amount of other organic solvent(s) may range, for example, from 1% to 30% by weight, preferably from 5% to 25% by weight and better still from 10% to 20% by weight relative to the total weight of the composition.

i) Sulfonic Polymer

The compositions according to the invention comprise at least one sulfonic polymer chosen from homopolymers of acrylamido-2-methylpropanesulfonic acid or salts thereof and copolymers of acrylamido-2-methylpropanesulfonic acid or salts thereof and of one or more nonionic monomers.

The sulfonic polymer may be crosslinked or non-crosslinked.

The sulfonic polymer may have a number-average molecular weight ranging from 1000 to 20 000 000 g/mol, preferably ranging from 20 000 to 5 000 000 and even more preferentially from 100 000 to 1 500 000 g/mol.

The compositions according to the invention can thus comprise at least one homopolymer of acrylamido-2-methylpropanesulfonic acid or salts thereof. More particularly, use is made of 2-acrylamido-2-methylpropanesulfonic acid, and also partially or totally neutralized forms thereof.

When the polymers are crosslinked, the crosslinking agents may be chosen from the polyolefinically unsaturated compounds commonly used for crosslinking polymers obtained by radical polymerization. Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allyl or vinyl ethers of polyfunctional alcohols, and also the allylic esters of phosphoric and/or vinylphosphonic acid derivatives, or mixtures of these compounds.

According to a preferred embodiment of the invention, the crosslinking agent is chosen from methylenebisacrylamide, allyl methacrylate or trimethylolpropane triacrylate (TMPTA). The degree of crosslinking generally ranges from 0.01 mol % to 10 mol % and more particularly from 0.2 mol % to 2 mol % relative to the polymer.

When the polymers used are homopolymers, they only comprise monomers containing a sulfonic group and, if they are crosslinked, one or more crosslinking agents.

The preferred 2-acrylamido-2-methylpropanesulfonic acid homopolymers are generally characterized in that they comprise, randomly distributed:

a) from 90% to 99.9% by weight of units of general formula (1) below:

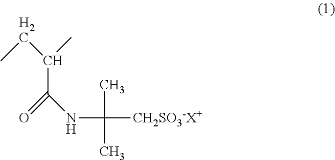

wherein X⁺ denotes a proton, a cation of an alkali metal such as sodium or potassium, a cation of an alkaline-earth metal such as calcium, or the ammonium ion, it being possible for at most 10 mol % of the cations X⁺ to be protons H⁺;
b) from 0.01% to 10% by weight of crosslinking units originating from at least one monomer containing at least two olefinic double bonds, the weight proportions being defined relative to the total weight of the polymer.

The homopolymers according to the invention that are more particularly preferred comprise from 98% to 99.5% by weight of units of formula (1) and from 0.2% to 2% by weight of crosslinking units.

Polymers of this type that may notably be mentioned include the crosslinked and neutralized 2-acrylamido-2-methylpropanesulfonic acid homopolymer sold by Clariant under the trade name Hostacerin AMPS® (CTFA name: ammonium polyacryldimethyltauramide).

The polymer may also be an amphiphilic homopolymer (or hydrophobic modified homopolymer) chosen from random amphiphilic 2-acrylamido-2-methylpropanesulfonic acid polymers modified by reaction with a C6-C22 n-monoalkylamine or di-n-alkylamine, such as those described in WO-A-00/31154, which are grafted homopolymers.

The compositions according to the invention can also comprise at least one copolymer of acrylamido-2-methylpropanesulfonic acid or salts thereof and of one or more nonionic monomers.

The AMPS® copolymers according to the invention may be crosslinked or non-crosslinked.

When the polymers are crosslinked, the crosslinking agents may be chosen from the polyolefinically unsaturated compounds commonly used for crosslinking polymers obtained by radical polymerization. Such agents are described above.

According to one preferred embodiment of the invention, the crosslinking agent is chosen from methylenebisacrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The copolymers according to the invention are obtained from AMPS® and from one or more hydrophilic or hydrophobic ethylenically unsaturated nonionic monomers and, if they are crosslinked, one or more crosslinking agents such as those defined above.

The 2-acrylamido-2-methylpropanesulfonic acid monomer of the copolymer contained in the composition in accordance with the invention is in free form or is partially or totally neutralized with a mineral base (sodium hydroxide, potassium hydroxide or aqueous ammonia) or an organic base, such as mono-, di- or triethanolamine, an aminomethylpropanediol, N-methylglucamine, basic amino acids, such as arginine and lysine, and also a mixture of these compounds.

Preferably, the 2-acrylamido-2-methylpropanesulfonic acid monomer according to the invention is partially or totally salified in the form of ammonium or sodium salt.

Preferably, the 2-acrylamido-2-methylpropanesulfonic acid monomer according to the invention is totally salified, preferably in the form of ammonium or sodium salt.

The AMPS® copolymers according to the invention contain one or more nonionic monomers chosen from water-soluble ethylenically unsaturated monomers, hydrophobic monomers, and mixtures thereof.

Among the nonionic water-soluble monomers, examples that may be mentioned include:
(meth)acrylamide,
N-vinylacetamide and N-methyl-N-vinylacetamide,
N-vinylformamide and N-methyl-N-vinylformamide,
maleic anhydride,
vinylamine,
N-vinyllactams including a cyclic alkyl group containing from 4 to 9 carbon atoms, such as N-vinylpyrrolidone, N-butyrolactam and N-vinylcaprolactam,
vinyl alcohol of formula CH₂═CHOH,
the water-soluble vinyl monomers of formula (2) below:

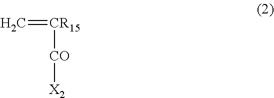

formula (2) wherein:
R₁₅ is chosen from H, —CH₃, —C₂H₅ and —C₃H₇,
X₂ is chosen from:
alkyl oxides of the type —OR₁₆ wherein Rib is a linear or branched, saturated or unsaturated hydrocarbon-based radical containing from 1 to 6 carbon atoms, optionally substituted with a halogen (iodine, bromine, chlorine or fluorine) atom; a hydroxyl (—OH) group; ether.

Mention is made, for example, of glycidyl (meth)acrylate, hydroxyethyl (meth)acrylate, and (meth)acrylates of ethylene glycol, of diethylene glycol or of polyalkylene glycol.

Preferably, the water-soluble monomer is chosen from acrylamide, vinylpyrrolidone and hydroxyalkyl (meth)acrylates, more particularly vinylpyrrolidone.

As copolymers of AMPS® in accordance with the invention with hydrophilic monomers, examples that may be mentioned include:
copolymers of acrylamido-2-methylpropanesulfonic acid and of vinylpyrrolidone, especially such as the commercial product Aristoflex AVC sold by Clariant,
crosslinked acrylamide/sodium acrylamido-2-methylpropanesulfonate copolymers, such as that used in the commercial product Sepigel 305® (INCI name: Polyacrylamide/C₁₃-C₁₄ isoparaffin/laureth-7) or that used in the commercial product sold under the name Simulgel 600® (INCI name: Acrylamide/sodium acryloyldimethyltaurate/isohexadecane/polysorbate-80®) by SEPPIC;
copolymers of AMPS® and of hydroxyethyl acrylate, for instance the sodium AMPS®/hydroxyethyl acrylate copolymer, such as that used in the commercial product sold under the name Simulgel NS® by SEPPIC (INCI name: Hydroxyethyl acrylate/sodium acryloyldimethyltaurate copolymer (and) squalane (and) polysorbate 60).

The concentration of AMPS® homopolymer or copolymer (i.e. of active material) generally ranges from 0.05% to 1% by weight relative to the total weight of the composition, and preferably from 0.05% to 0.8% by weight and even more particularly from 0.1% to 0.5% by weight.

ii) Crosslinked Copolymer of Acrylic Acid and of C₁₀-C₃₀Alkyl Acrylate

The composition according to the invention comprises at least one crosslinked copolymer of acrylic acid and of C₁₀-C₃₀ alkyl acrylate.

The acrylic acid monomer is preferably present in amounts ranging from 60% to 95% by weight relative to the total weight of the copolymer.

The C₁₀-C₃₀ alkyl acrylate monomer is preferably present in amounts ranging from 1% to 50% by weight and more particularly from 4% to 40% by weight relative to the total weight of the copolymer.

The crosslinked copolymer of acrylic acid and of a C10-C30 alkyl acrylate those comprising a) at least one hydrophilic unit of unsaturated olefinic carboxylic acid type, and ib) at least one hydrophobic unit of C₁₀-C₃₀ alkyl ester of unsaturated carboxylic acid type. Mention may be made of the C₁₀-C₃₀ alkyl esters of carboxylic acids of the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate. Polymers of this type are described and prepared, for example, according to patents U.S. Pat. Nos. 3,915,921 and 4,509,949.

Among associative polymers of this type, use will be made more particularly of those constituted of 95% to 60% by weight of acrylic acid (hydrophilic unit), 4% to 40% by weight of C₁₀-C₃₀ alkyl acrylate (hydrophobic unit), and 0 to 6% by weight of crosslinking polymerizable monomer, or else those constituted of 98% to 96% by weight of acrylic acid (hydrophilic unit), 1 to 4% by weight of C₁₀-C₃₀ alkyl acrylate (hydrophobic unit), and 0.1% to 0.6% by weight of polymerizable crosslinking monomer, such as those described above. The copolymer is typically partially or totally crosslinked by at least one conventional crosslinking agent. The crosslinking agents are notably polyunsaturated compounds. These compounds are in particular diallyl phthalates, divinylbenzene, allyl (meth)acrylate, (poly)ethylene glycol di(meth)acrylate or methylenebisacrylamide. The content of crosslinking agent ranges from 0 to 6% by weight and preferably from 0.001% to 6% by weight relative to the total weight of the copolymer.

Among said abovementioned polymers, the ones that are most particularly preferred are the products sold by Lubrizol under the trade names Pemulen TR1®, Pemulen TR2®, Carbopol 1382®, Carbopol ETD 2020®, Carbopol Ultrez 20® and Carbopol Ultrez 21® (INCI name: Acrylates/C10-30 alkyl acrylate crosspolymer), and even more preferentially Pemulen TR1 and the product sold by SEPPIC under the name Coatex SX®, and Carbopol Ultrez 21.

The concentration of crosslinked copolymer (i.e. of active material) generally ranges from 0.05% to 1% by weight relative to the total weight of the composition, and preferably from 0.1% to 0.8% by weight and even more particularly from 0.2% to 0.6% by weight.

iii) Additional Organic Thickening Polymer

According to one particular embodiment of the invention, the composition also comprises iii) one or more organic thickening polymers other than the polymers i) and ii) as defined above.

The term “thickening polymer” is intended to mean a polymer which, when introduced at 1% by weight in an aqueous solution or an aqueous-alcoholic solution containing 30% ethanol, and at pH=7, or in an oil chosen from liquid petroleum jelly, isopropyl myristate or cyclopentadimethylsiloxane, makes it possible to achieve a viscosity of at least 100 cps and preferably of at least 500 cps, at 25° C. and at a shear rate of 1 s⁻¹. This viscosity may be measured using a cone/plate viscometer (Haake R600 rheometer or the like). The thickening polymers may thicken the aqueous phase and/or the fatty phase, preferentially the aqueous phase.

The term “organic” thickening polymer is intended to mean a thickening polymer as defined previously, which is formed from carbon and hydrogen, and possibly nitrogen, oxygen, sulfur, halogens such as fluorine, chlorine or bromine, and also phosphorus, alkali metals such as sodium or potassium, or alkaline-earth metals such as magnesium or calcium. The organic polymers according to the invention do not comprise silicon.

The expression “non-cellulose-based organic thickening polymer” is intended to mean an organic thickening polymer not comprising any cellulose units.

The organic thickening polymers according to the invention may be of natural or synthetic origin, preferably natural.

The thickening polymers may be associative or nonassociative anionic, cationic, amphoteric or nonionic polymers.

They may be thickeners for the aqueous or oily phases.

By way of aqueous-phase thickening polymers, mention may be made of associative or nonassociative, preferably nonassociative, thickening polymers, comprising sugar units.

For the purposes of the present invention, the term “sugar” unit is intended to mean a unit derived from a carbohydrate of formula Cₙ(H₂O)ₙ₋₁ or (CH₂O)ₙ, which may be optionally modified by substitution and/or by oxidation and/or by dehydration.

The sugar units which can participate in the composition of the thickening polymers of the invention preferably result from the following sugars: glucose, galactose, arabinose, rhamnose, mannose, xylose, fucose, anhydrogalactose, galacturonic acid, glucuronic acid, mannuronic acid, galactose sulfate, anhydrogalactose sulfate and fructose.

Thickening polymers of the invention that may in particular be mentioned include native gums such as:

a) tree or shrub exudates, including:
gum arabic (branched polymer of galactose, arabinose, rhamnose and glucuronic acid);
ghatti gum (polymer derived from arabinose, galactose, mannose, xylose and glucuronic acid);
karaya gum (polymer derived from galacturonic acid, galactose, rhamnose and glucuronic acid);
gum tragacanth (polymer of galacturonic acid, galactose, fucose, xylose and arabinose);

b) gums derived from algae, including:
agar (polymer derived from galactose and anhydrogalactose);
alginates (polymers of mannuronic acid and of glucuronic acid);
carrageenans and furcellerans (polymers of galactose sulfate and of anhydrogalactose sulfate);

c) gums derived from seeds or tubers, including:
guar gum (polymer of mannose and galactose);
locust bean gum (polymer of mannose and galactose);
fenugreek gum (polymer of mannose and galactose);
tamarind gum (polymer of galactose, xylose and glucose);
konjac gum (polymer of glucose and mannose);

d) microbial gums, including:
xanthan gum (polymer of glucose, mannose acetate, mannose/pyruvic acid and glucuronic acid);
gellan gum (polymer of partially acylated glucose, rhamnose and glucuronic acid);
scleroglucan gum (glucose polymer);

e) plant extracts, including:
cellulose (glucose polymer);
starch (glucose polymer); and
inulin.

These polymers can be physically or chemically modified. As physical treatment, mention may in particular be made of the temperature.

As chemical treatments, mention may be made of esterification, etherification, amidation and oxidation reactions. These treatments make it possible to lead to polymers that may in particular be nonionic, anionic or amphoteric.

Preferably, these chemical or physical treatments are applied to guar gums, locust bean gums, starches and celluloses.

The nonionic guar gums which can be used according to the invention can be modified by (poly)hydroxy(C₁-C₆)alkyl groups.

Mention may be made, by way of example, among the (poly)hydroxy(C₁-C₆)alkyl groups, of the hydroxymethyl, hydroxyethyl, hydroxypropyl and hydroxybutyl groups.

These guar gums are well known from the prior art and may be prepared, for example, by reacting corresponding alkene oxides, for instance propylene oxides, with the guar gum so as to obtain a guar gum modified with hydroxypropyl groups.

The degree of hydroxyalkylation preferably ranges from 0.4 to 1.2 and corresponds to the number of alkylene oxide molecules consumed by the number of free hydroxyl functional groups present on the guar gum.

Such nonionic guar gums optionally modified with hydroxyalkyl groups are sold, for example, under the trade names Jaguar HP8, Jaguar HP60 and Jaguar HP120 by Rhodia Chimie.

The botanical origin of the starch molecules used in the present invention may be cereals or tubers. Thus, the starches are, for example, chosen from corn starch, rice starch, cassava starch, barley starch, potato starch, wheat starch, sorghum starch or pea starch.

The starches can be chemically or physically modified, in particular by one or more of the following reactions: pregelatinization, oxidation, crosslinking, esterification, etherification, amidation or heat treatments.

Distarch phosphates or compounds rich in distarch phosphate will preferentially be used, for instance the product sold under the references Prejel VA-70-T AGGL (gelatinized hydroxypropyl cassava distarch phosphate), Prejel TK1 (gelatinized cassava distarch phosphate) and Prejel 200 (gelatinized acetylated cassava distarch phosphate) by Avebe, or Structure Zea from National Starch (gelatinized corn distarch phosphate).

According to the invention, use may also be made of amphoteric starches, these amphoteric starches comprising one or more anionic groups and one or more cationic groups. The anionic and cationic groups may be bonded to the same reactive site of the starch molecule or to different reactive sites; they are preferably bonded to the same reactive site. The anionic groups may be of carboxylic, phosphate or sulfate type, preferably carboxylic type. The cationic groups may be of primary, secondary, tertiary or quaternary amine type.

The starch molecules may be derived from any plant source of starch, notably such as corn, potato, oat, rice, tapioca, sorghum, barley or wheat. It is also possible to use hydrolysates of the starches mentioned above. The starch is preferably derived from potato.

The nonassociative thickening polymers of the invention can be cellulose-based polymers not comprising C₁₀-C₃₀ fatty chains in their structure.

According to the invention, the term “cellulose-based polymer” is intended to mean any polysaccharide compound having in its structure sequences of glucose residues linked together via β-1,4 bonds; in addition to unsubstituted celluloses, the cellulose derivatives may be anionic, cationic, amphoteric or nonionic.

Thus, the cellulose-based polymers of the invention may be chosen from unsubstituted celluloses, including those in a microcrystalline form, and cellulose ethers.

Among these cellulose-based polymers, cellulose ethers, cellulose esters and cellulose ether esters are distinguished.

Among the cellulose esters are mineral esters of cellulose (cellulose nitrates, sulfates or phosphates, etc.), organic cellulose esters (cellulose monoacetates, triacetates, amidopropionates, acetatebutyrates, acetatepropionates or acetatetrimellitates, etc.), and mixed organic/mineral esters of cellulose, such as cellulose acetatebutyrate sulfates and cellulose acetatepropionate sulfates. Among the cellulose ether esters, mention may be made of hydroxypropylmethylcellulose phthalates and ethylcellulose sulfates.

Among the nonionic cellulose ethers without a C₁₀-C₃₀ fatty chain, i.e. which are “nonassociative”, mention may be made of (C₁-C₄)alkylcelluloses, such as methylcelluloses and ethylcelluloses (for example, Ethocel standard 100 Premium from Dow Chemical); (poly)hydroxy(C₁-C₄)alkylcelluloses, such as hydroxymethylcelluloses, hydroxyethylcelluloses (for example, Natrosol 250 HHR provided by Aqualon) and hydroxypropylcelluloses (for example, Klucel EF from Aqualon); mixed (poly)hydroxy(C₁-C₄)alkyl-(C₁-C₄)alkylcelluloses celluloses, such as hydroxypropylmethylcelluloses (for example, Methocel E4M from Dow Chemical), hydroxyethyl methylcelluloses, hydroxyethylethylcelluloses (for example, Bermocoll E 481 FQ from Akzo Nobel) and hydroxybutylmethylcelluloses.

Among the anionic cellulose ethers without a fatty chain, mention may be made of (poly)carboxy(C₁-C₄)alkylcelluloses and salts thereof. Examples that may be mentioned include carboxymethylcelluloses, carboxymethylmethylcelluloses (for example Blanose 7M from Aqualon) and carboxymethylhydroxyethylcelluloses, and the sodium salts thereof.

Among the cationic cellulose ethers without a fatty chain, mention may be made of cationic cellulose derivatives such as cellulose copolymers or cellulose derivatives grafted with a water-soluble quaternary ammonium monomer, and notably described in patent U.S. Pat. No. 4,131,576, such as (poly)hydroxy(C₁-C₄)alkylcelluloses, for instance hydroxymethyl-, hydroxyethyl- or hydroxypropylcelluloses notably grafted with a methacryloylethyltrimethylammonium, methacrylamidopropyltrimethylammonium or dimethyldiallylammonium salt. The commercial products corresponding to this definition are more particularly the products sold under the names Celquat® L 200 and Celquat® H 100 by National Starch.

According to one particular embodiment of the invention, the thickening polymer(s) of the invention result from the (co)polymerization of acrylate monomer CH₂═C(R′)—COOR′″ (VIa) and/or from acrylamide monomer CH₂═C(R′)—CO—N(R″)-L-Y⁻ M⁺ (VIb); in said formulae (VIa) and (VIb), R′ and R″, which may be identical or different, representing a hydrogen atom or a (C₁-C₆) alkyl group such as methyl, preferably hydrogen, R′″ represents an alkali metal, an alkaline-earth metal, a hydrogen atom or a (C₁-C₆) alkyl group optionally substituted in particular with one or more hydroxyl, carboxy or amino groups, preferably R′″ represents a hydrogen atom, L representing a cyclic or acylic, saturated or unsaturated, linear or branched, divalent hydrocarbon-based group, optionally interrupted by one or more heteroatoms such as O or N and comprising from 1 to 20 carbon atoms, preferably from 1 to 6 carbon atoms, preferably L represents the divalent group —[C(R′)(R″)]ₚ— with p representing an integer between 1 and 4, preferably 2 and 3, such as 2, R′ and R″ being as defined above, more particularly L represents —C(R′)(R″)—CH₂— or —CH₂—C(R′)(R″)— with R′ and R″ as defined above, preferably R′ and R″ represent a (C₁-C₄) alkyl group such as methyl; Y⁻ represents an anionic group such as carboxylate, phosphate or phosphonate, and M⁺ being a cationic counterion, preferably an alkali metal, such as sodium, it being possible for said copolymer to be in a direct or inverse emulsion, preferably an inverse emulsion. More preferentially, the thickening polymer(s) of the invention result from the copolymerization of acrylate monomer CH₂═C(R′)—COOH (VIa) and of acrylamide monomer CH₂═C(R′)—CO—N(R″)-L-Y⁻ M⁺ (VIb) as defined above.

Among the nonassociative thickening polymers without sugar units that can be used, mention may be made of ammonium acrylate homopolymers or copolymers of ammonium acrylate and of acrylamide, alone or as mixtures.

A first family of nonassociative thickening polymers that is suitable for use is represented by crosslinked acrylic acid homopolymers.

Among the homopolymers of this type, mention may be made of those crosslinked with an allyl alcohol ether of the sugar series, for instance the products sold under the names Carbopol 980, 981, 954, 2984 and 5984 by Noveon or the products sold under the names Synthalen M and Synthalen K by 3 VSA.

The nonassociative thickening polymers may also be crosslinked (meth)acrylic acid copolymers, such as the polymer sold under the name Aqua SF1 by Noveon.

The composition may similarly comprise, as nonassociative thickening polymers, ammonium acrylate homopolymers or copolymers of ammonium acrylate and of acrylamide.

As examples of ammonium acrylate homopolymers, mention may be made of the product sold under the name Simulgel 600 acrylamide/sodium acryloyldimethyltaurate copolymer isohexadecane and polysorbate 80 sold by SEPPIC, and Microsap PAS 5193 by Hoechst. Among the copolymers of ammonium acrylate and of acrylamide, mention may be made of the product sold under the name Bozepol C Nouveau or the product PAS 5193 sold by Hoechst. Reference may be made notably to FR 2 416 723, U.S. Pat. Nos. 2,798,053 and 2,923,692 as regards the description and preparation of such compounds.

Among the aqueous-phase thickening polymers, mention may also be made of the non-cellulose-based associative polymers that are well known to those skilled in the art and in particular of nonionic, anionic, cationic or amphoteric nature.

It is recalled that “associative polymers” are polymers that are capable, in an aqueous medium, of reversibly associating with each other or with other molecules.

Their chemical structure more particularly comprises at least one hydrophilic zone and at least one hydrophobic zone.

The term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Mention may be made, among the associative polymers of anionic type, of:
(a) those including at least one hydrophilic unit and at least one fatty-chain allyl ether unit, more particularly those of which the hydrophilic unit is constituted by an ethylenic unsaturated anionic monomer, more particularly still a vinylcarboxylic acid and most particularly an acrylic acid or a methacrylic acid or mixtures thereof.

Among these anionic associative polymers, those that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether, and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), especially those sold by CIBA under the names Salcare SC80® and Salcare SC90®, which are aqueous 30% emulsions of a crosslinked terpolymer of methacrylic acid, of ethyl acrylate and of steareth-10 allyl ether (40/50/10).

Mention may also be made of the acrylic acid/lauryl methacrylate/vinylpyrrolidone terpolymer sold under the name Acrylidone LM by ISP.
(C) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by Newphase Technologies.
(d) acrylic terpolymers comprising:

i) approximately 20% to 70% by weight of an α,β-monoethylenically unsaturated carboxylic acid [A],

ii) approximately 20% to 80% by weight of an α,β-monoethylenically unsaturated non-surfactant monomer other than [A],

iii) about 0.5% to 60% by weight of a nonionic monourethane which is the product of reaction of a monohydric surfactant with a monoethylenically unsaturated monoisocyanate,

such as those described in patent application EP-A-0 173 109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
(e) copolymers comprising among their monomers an α,β-monoethylenically unsaturated carboxylic acid and an ester of an α,β-monoethylenically unsaturated carboxylic acid and of an oxyalkylenated fatty alcohol.

Preferentially, these compounds also comprise as monomer an ester of an α,β-monoethylenically unsaturated carboxylic acid and of a C₁-C₄ alcohol.

An example of a compound of this type that may be mentioned is Aculyn 22® sold by Röhm & Haas, which is a methacrylic acid/ethyl acrylate/oxyalkylenated stearyl methacrylate terpolymer.
(f) amphiphilic polymers including at least one ethylenically unsaturated monomer bearing a sulfonic group, in free or partially or totally neutralized form and comprising at least one hydrophobic part. These polymers may be crosslinked or non-crosslinked. They are preferably crosslinked.

According to one particular embodiment of the invention, the polymer(s) iii) are associative, in particular cationic. Mention may be made of:
(I) cationic associative polyurethanes;
(II) the compound sold by Noveon under the name Aqua CC and which corresponds to the INCI name Polyacrylate-1 Crosspolymer.

Polyacrylate-1 Crosspolymer is the product of polymerization of a monomer mixture comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl) methacrylate, and
an ethylene glycol dimethacrylate.
(III) quaternized (poly)hydroxyethylcelluloses modified with groups including at least one fatty chain, such as alkyl, arylalkyl or alkylaryl groups including at least 8 carbon atoms, or mixtures thereof. The alkyl radicals borne by the above quaternized celluloses or hydroxyethylcelluloses preferably include from 8 to 30 carbon atoms. The aryl radicals preferably denote phenyl, benzyl, naphthyl or anthryl groups. Examples of quaternized alkylhydroxyethylcelluloses containing C₈-C₃₀ fatty chains that may be indicated include the products Quatrisoft LM 200®, Quatrisoft LM-X 529-18-A®, Quatrisoft LM-X 529-18-B® (C₁₂ alkyl) and Quatrisoft LM-X 529-8® (C₁₈ alkyl) sold by Aqualon, and the products Crodacel QM®, Crodacel QLO (C₁₂ alkyl) and Crodacel QS® (C₁₈ alkyl) sold by Croda and the product Softcat SL 100® sold by Aqualon.
(IV) cationic polyvinyllactam polymers.

Such polymers are described, for example, in patent application WO-00/68282.

As cationic poly(vinyllactam) polymers according to the invention, vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoyldimethylmethacrylamidopropylammonium tosylate terpolymers, vinylpyrrolidone/dimethylaminopropylmethacrylamide/lauryldimethylmethacrylamidopropyl ammonium tosylate or chloride terpolymers are notably used.

According to another particular embodiment, the polymer(s) iii) are amphoteric associative polymer(s). They are preferably chosen from those including at least one noncyclic cationic unit. Even more particularly, those prepared from or comprising 1 to 20 mol %, preferably 1.5 to 15 mol % and even more particularly 1.5 to 6 mol % of fatty-chain monomer relative to the total number of moles of monomers are preferred.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

The associative polymers of nonionic type that may be used according to the invention are preferably chosen from:

(a) copolymers of vinylpyrrolidone and of fatty-chain hydrophobic monomers, of which examples that may be mentioned include:
the products Antaron V216® or Ganex V216® (vinylpyrrolidone/hexadecene copolymer), sold by ISP;
the products Antaron V220® or Ganex V220® (vinylpyrrolidone/eicosene copolymer), sold by ISP;

(b) copolymers of C₁-C₆ alkyl methacrylates or acrylates and of amphiphilic monomers including at least one fatty chain, for instance the oxyethylated methyl acrylate/stearyl acrylate copolymer sold by Goldschmidt under the name Antil 208®;

(c) copolymers of hydrophilic methacrylates or acrylates and of hydrophobic monomers including at least one fatty chain, for instance the polyethylene glycol methacrylate/lauryl methacrylate copolymer;

(d) polyurethane polyethers including in their chain both hydrophilic blocks usually of polyoxyethylenated nature and hydrophobic blocks, which may be aliphatic sequences alone and/or cycloaliphatic and/or aromatic sequences;

(e) polymers with an aminoplast ether backbone containing at least one fatty chain, such as the Pure Thix® compounds sold by Sud-Chemie;

(f) celluloses or derivatives thereof, modified with groups including at least one fatty chain, such as alkyl, arylalkyl or alkylaryl groups or mixtures thereof wherein the alkyl groups are of C₈, and in particular:
nonionic alkylhydroxyethylcelluloses such as the products Natrosol Plus Grade 330 CS and Polysurf 67 (C₁₆ alkyl) sold by Aqualon;
nonionic nonoxynylhydroxyethylcelluloses such as the product Amercell HM-1500 sold by Amerchol;
nonionic alkylcelluloses, such as the product Bermocoll EHM 100 sold by Berol Nobel;

(g) associative guar derivatives, for instance hydroxypropyl guars modified with a fatty chain, such as the product Esaflor HM 22 (modified with a C₂₂ alkyl chain) sold by Lamberti; the product Miracare XC 95-3 (modified with a C₁₄ alkyl chain) and the product RE 205-146 (modified with a C₂₀ alkyl chain) sold by Rhodia Chimie.

Preferably, the polyurethane polyethers include at least two hydrocarbon-based lipophilic chains containing from 6 to 30 carbon atoms, separated by a hydrophilic block, the hydrocarbon-based chains possibly being side chains or chains at the end of the hydrophilic block. In particular, it is possible for one or more side chains to be envisaged. In addition, the polymer may include a hydrocarbon-based chain at one end or at both ends of a hydrophilic block.

The polyurethane polyethers may be multiblock, in particular in triblock form. The hydrophobic blocks may be at each end of the chain (for example: triblock copolymer bearing a hydrophilic central block) or distributed both at the ends and in the chain (for example, multiblock copolymer). These same polymers may also be graft polymers or star polymers.

The nonionic fatty-chain polyurethane polyethers may be triblock copolymers, the hydrophilic block of which is a polyoxyethylene chain including from 50 to 1000 oxyethylene groups. The nonionic polyurethane polyethers include a urethane bond between the hydrophilic blocks, whence the origin of the name.

By extension, also included among the nonionic fatty-chain polyurethane polyethers are those wherein the hydrophilic blocks are linked to the lipophilic blocks via other chemical bonds.

As examples of nonionic fatty-chain polyurethane polyethers that may be used in the invention, use may also be made of Rheolate 205® bearing a urea function, sold by Rheox, or Rheolate® 208, 204 or 212, and also Acrysol RM 184®.

Mention may also be made of the product Elfacos T210® bearing a C₁₂-C₁₄ alkyl chain, and the product Elfacos T212® bearing a C₁₈ alkyl chain, from Akzo.

The product DW 1206B® from Röhm & Haas bearing a 020 alkyl chain and a urethane bond, sold at a solids content of 20% in water, may also be used.

Use may also be made of solutions or dispersions of these polymers, notably in water or in aqueous-alcoholic medium. Examples of such polymers that may be mentioned include Rheolate® 255, Rheolate® 278 and Rheolate® 244 sold by Rheox. Use may also be made of the products DW 1206F and DW 1206J sold by Röhm & Haas.

The polyurethane polyethers that may be used according to the invention are in particular those described in the article by G. Fonnum, J. Bakke and Fk. Hansen—Colloid Polym. Sci., 271, 380-389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are notably sold by Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Use may also be made of fatty-phase-thickening polymers.

Preferably, the polymers for structuring the oily phase via physical interactions are chosen from polyamides, silicone polyamides, saccharide or polysaccharide mono- or polyalkyl esters, N-acylamino acid amide derivatives, and copolymers comprising an alkylene or styrene block, these copolymers possibly being diblock, triblock, multiblock or radial-block polymers, also known as star copolymers, or alternatively comb polymers.

1) Polymers Bearing at Least One Crystallizable Block in the Backbone

These are also polymers that are soluble or dispersible in the oil or oily phase by heating above their melting point m.p. These polymers are in particular block copolymers constituted of at least 2 blocks of different chemical nature, one of which is crystallizable.

As polymers bearing in the backbone at least one crystallizable block that are suitable for use in the invention, mention may be made of:

i). the polymers defined in document U.S. Pat. No. 5,156,911;

ii). block copolymers of olefin or of cycloolefin containing a crystallizable chain, for instance those derived from the block polymerization of:
cyclobutene, cyclohexene, cyclooctene, norbornene (i.e. bicyclo(2.2.1)hept-2-ene), 5-methylnorbornene, 5-ethylnorbornene, 5,6-dimethylnorbornene, 5,5,6-trimethylnorbornene, 5-ethylidenenorbornene, 5-phenylnorbornene,

5-benzylnorbornene, 5-vinylnorbornene, 1,4,5,8-dimethano-1,2,3,4,4a,5,8a-octahydronaphthalene, dicyclopentadiene, and mixtures thereof;
with ethylene, propylene, 1-butene, 3-methyl-1-butene, 1-hexene, 4-methyl-1-pentene, 1-octene, 1-decene or 1-eicosene, or mixtures thereof. These block copolymers may be in particular (ethylene/norbornene) block copolymers and (ethylene/propylene/ethylidenenorbornene) block terpolymers.

Those resulting from the block copolymerization of at least 2 C₂-C₁₆, and better still C₂-C₁₂, α-olefins such as those mentioned above and in particular block bipolymers of ethylene and of 1-octene may also be used.

Copolymers containing at least one crystallizable block, the rest of the copolymer being amorphous (at ambient temperature). These copolymers may also contain two crystallizable blocks of different chemical nature. The preferred copolymers are those that simultaneously contain at ambient temperature a crystallizable block and an amorphous block that are both hydrophobic and lipophilic, sequentially distributed; mention may be made, for example, of polymers containing one of the crystallizable blocks and one of the amorphous blocks below:
block that is crystallizable by nature: a) of polyester type, for instance poly(alkylene terephthalate), b) of polyolefin type, for instance polyethylenes or polypropylenes;
amorphous and lipophilic block, for instance, amorphous polyolefins or copoly(olefin)s such as poly(isobutylene), hydrogenated polybutadiene or hydrogenated poly(isoprene).

As examples of such copolymers containing a crystallizable block and an amorphous block, mention may be made of:

a) poly(δ-caprolactone)-b-poly(butadiene) block copolymers, preferably used hydrogenated, such as those described in the article “Melting behavior of poly(δ-caprolactone)-block-polybutadiene copolymers” from S. Nojima, Macromolecules, 32, 3727-3734 (1999),

b) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),

c) the poly(ethylene)-b-copoly(ethylene/propylene) block copolymers cited in the articles “Morphology of semi-crystalline block copolymers of ethylene(ethylene-alt-propylene)” by P. Rangarajan et al., Macromolecules, 26, 4640-4645 (1993) and “Polymer aggregates with crystalline cores: the system poly(ethylene)poly(ethylene-propylene)” P. Richter et al., Macromolecules, 30, 1053-1068 25 (1997),

d) the poly(ethylene)-b-poly(ethylethylene) block copolymers mentioned in the general article “Crystallization in block copolymers” by I. W. Hamley, Advances in Polymer Science, vol 148, 113-137 (1999).

The semicrystalline polymers that may be used in the context of the invention may be non-crosslinked or partially crosslinked, provided that the degree of crosslinking does not impede their dissolution or dispersion in the liquid oily phase by heating above their melting point. It may then be a case of chemical crosslinking, by reaction with a multifunctional monomer during the polymerization. It may also be a case of physical crosslinking, which may then be due either to the establishment of bonds of hydrogen or dipolar type between groups borne by the polymer, for instance dipolar interactions between carboxylate ionomers, these interactions being in small amount and borne by the polymer backbone; or due to a phase separation between the crystallizable blocks and the amorphous blocks borne by the polymer.

Preferably, the semicrystalline polymers that are suitable for the invention are non-crosslinked.

As particular examples of semicrystalline polymers that may be used in the composition according to the invention, mention may be made of the Intelimer® products from Landec described in the brochure “Intelimer® polymers”. These polymers are in solid form at ambient temperature (25° C.). They bear crystallizable side chains and contain the monomer. Mention may be made especially of Landec IP22®, with a melting point m.p. of 56° C., which is a viscous, impermeable, non-tacky product at ambient temperature.

It is also possible to use the semicrystalline polymers described in Examples 3, 4, 5, 7 and 9 of document U.S. Pat. No. 5,156,911, resulting from the copolymerization of acrylic acid and of C₅ to C₁₆ alkyl (meth)acrylate, such as those resulting from the copolymerization:
of acrylic acid, of hexadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio,
of acrylic acid and of octadecyl methacrylate in a 2.5/97.5 ratio.

It is also possible to use the polymer “Structure O” sold by National Starch, such as the product described in document U.S. Pat. No. 5,736,125, of m.p. 44° C., and also semicrystalline polymers containing crystallizable side chains comprising fluoro groups as described in Examples 1, 4, 6, 7 and 8 of document WO-A-01/19333.

It is also possible to use the semicrystalline polymers obtained by copolymerization of stearyl acrylate and of acrylic acid or of NVP, or by copolymerization of behenyl acrylate and of acrylic acid or NVP, as described in document U.S. Pat. No. 5,519,063 or EP-A-0 550 745.

According to one particular embodiment variant, the semicrystalline polymers that are suitable for use in the present invention are especially alkyl acrylates, among which mention may be made of the Landec copolymers:
Doresco IPA 13-1®: polystearyl acrylate, m.p. of 49° C. and MW of 145 000;
Doresco IPA 13-3®: polyacrylate/methacrylic acid, m.p. of 65° C. and MW of 114 000;
Doresco IPA 13-4®: polyacrylate/vinylpyrrolidone, m.p. of 44° C. and MW of 387 000;
Doresco IPA13-5®: polyacrylate/hydroxyethyl methacrylate, m.p. of 47° C. and MW of 397 600;
Doresco IPA 13-6®: polybehenyl acrylate, m.p. of 66° C.

2) Non-Silicone Polyamides

The particular polyamides used in the composition according to the invention are preferably those described in document U.S. Pat. No. 5,783,657 from Union Camp. The section of U.S. Pat. No. 5,783,657 devoted to these polymers is incorporated by reference.

Each of these polyamides satisfies in particular formula (V) below:

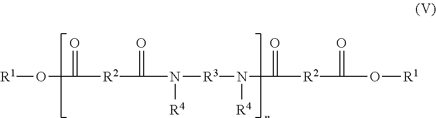

formula (V) wherein:
n denotes a whole number of amide units such that the number of ester groups represents from 10% to 50% of the total number of ester and amide groups;
R¹ is independently in each case an alkyl or alkenyl group containing at least 4 carbon atoms and especially from 4 to 24 carbon atoms;
R² independently represents, in each case, a C₄ to C₅₅ hydrocarbon-based group, on condition that at least 50% of the R₂ groups represent a C₃₀ to C₅₅ hydrocarbon-based group;
R³ independently represents, in each case, an organic group bearing at least two carbon atoms, hydrogen atoms and optionally one or more oxygen or nitrogen atoms; and
R⁴ independently represents, in each case, a hydrogen atom, a C₁ to C₁₀ alkyl group or a direct bond to R₃ or to another R₄ so that the nitrogen atom to which both R₃ and R₄ are attached are part of a heterocyclic structure defined by R₄—NR₃, with at least 50% of the R₄ groups representing a hydrogen atom.

In particular, the ester groups of this polyamide represent from 15% to 40% and at best from 20% to 35% of the total number of ester and amide groups. Furthermore, n advantageously represents an integer ranging from 1 to 10 and better still from 1 to 5, limits inclusive.

Preferably, R¹ is a C₁₂ to C₂₂ and preferably C₁₆ to C₂₂ alkyl group. Advantageously, R² can be a C₁₀ to C₄₂ hydrocarbon-based (alkylene) group. Preferably, at least 50% and better still at least 75% of the R² groups are groups containing from 30 to 42 carbon atoms. The other R² groups are C₄ to C₁₉ and preferably C₄ to C₁₂ hydrogen-containing groups. Preferably, R³ represents a C₂ to C₃₆ hydrocarbon-based group or a polyoxyalkylene group and R⁴ represents a hydrogen atom. Preferably, R³ represents a C₂ to C₁₂ hydrocarbon-based group. The hydrocarbon-based groups may be linear, cyclic or branched, and saturated or unsaturated groups. Moreover, the alkyl and alkylene groups may be linear or branched, and saturated or unsaturated groups.

The thickening of the liquid fatty phase may be obtained by means of one or more polyamides defined above. In general, these polyamides are in the form of mixtures, these mixtures also possibly containing a synthetic product corresponding to a polyamide as defined above with n being 0, i.e. a diester.

As structuring polyamides that may be used in the invention, mention may also be made of polyamide resins resulting from the condensation of an aliphatic dicarboxylic acid and a diamine (including compounds containing more than two carboxyl groups and two amine groups), the carboxyl and amine groups of adjacent individual units being condensed in the form of an amide bond. These polyamide resins are especially the products sold under the brand name Versamid® by the companies General Mills, Inc. and Henkel Corp., under the brand name Onamid®, especially Onamid® S or C. These resins have a weight-average molecular mass ranging from 6000 to 9000. For further information regarding these polyamides, reference may be made to U.S. Pat. Nos. 3,645,705 and 3,148,125. Use is made more especially of Versamid® 30 or 744.

2) Saccharide or Polysaccharide Mono- or Polyalkyl Esters

Among the saccharide or polysaccharide monoalkyl or polyalkyl esters that are suitable for use in the invention, mention may be made of dextrin or inulin alkyl or polyalkyl esters.

They may in particular be a mono- or polyester of dextrin and of at least one fatty acid corresponding in particular to formula (VI) below:

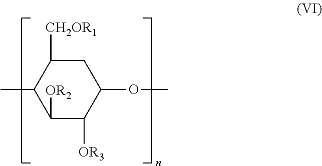

formula (VI) wherein:
n is an integer ranging from 3 to 200, especially ranging from 20 to 150 and in particular ranging from 25 to 50,
R₁, R₂ and R₃, which may be identical or different, are chosen from hydrogen and an acyl group (R—C(O)—) wherein the R radical is a linear or branched, saturated or unsaturated hydrocarbon-based group containing from 7 to 29, in particular from 7 to 21, especially from 11 to 19, more particularly from 13 to 17, or even 15, carbon atoms, with the proviso that at least one of said R₁, R₂ or R₃ radicals is other than hydrogen.

In particular, R₁, R₂ and R₃ may represent hydrogen or an acyl group (R—C(O)—) wherein R is a hydrocarbon-based radical as defined previously, with the proviso that at least two of said radicals R₁, R₂ or R₃ are identical and other than hydrogen.

The R₁, R₂ and R₃ radicals may all contain an acyl group (R—C(O)), which may be identical or different and especially identical.

In particular, n mentioned above advantageously ranges from 25 to 50 and is especially equal to 38 in the general formula of the saccharide ester that may be used in the present invention.

When the R₁, R₂ and/or R₃ radicals, which may be identical or different, contain an acyl group (R—C(O)), these radicals may be chosen especially from caprylic, capric, lauric, myristic, palmitic, stearic, arachic, behenic, isobutyric, isovaleric, 2-ethylbutyric, ethylmethylacetic, isoheptanoic, 2-ethylhexanoic, isononanoic, isodecanoic, isotridecanoic, isomyristic, isopalmitic, isostearic, isoarachic, isohexanoic, decenoic, dodecenoic, tetradecenoic, myristoleic, hexadecenoic, palmitoleic, oleic, elaidic, asclepinic, gondoleic, eicosenoic, sorbic, linoleic, linolenic, punicic, stearidonic, arachidonic and stearolic radicals, and mixtures thereof.

Preferably, at least one dextrin palmitate is used as fatty acid ester of dextrin. This ester may be used alone or as a mixture with other esters.

Advantageously, the fatty acid ester of dextrin has a degree of substitution of less than or equal to 2.5, especially ranging from 1.5 to 2.5 and preferably from 2 to 2.5 on the basis of one glucose unit. The weight-average molecular weight of the dextrin ester may in particular be from 10 000 to 150 000, especially from 12 000 to 100 000 and even from 15 000 to 80 000.

Dextrin esters, in particular dextrin palmitates, are commercially available under the name Rheopearl TL or Rheopearl KL by Chiba Flour.

3) N-Acylamino Acid Amide Derivatives

The N-acylamino acid amides that may be used are, for example, diamides from the combination of an N-acylamino acid with amines comprising from 1 to 22 carbon atoms, such as those described in document FR 2 281 162. They are, for example, alkylglutamic acid amide derivatives such as the lauroylglutamic acid dibutylamide sold by Ajinomoto under the name Gelling Agent GP-1, or alternatively the 2-ethylhexanoylglutamic acid dibutylamide sold by Ajinomoto under the name Gelling Agent GA-01.

Among the fatty-phase thickening polymers, polymers bearing in the backbone at least one crystallizable block are preferred.

The aqueous-phase or fatty-phase thickening polymers may be used alone or as mixtures in all proportions.

Preferably, the thickeners are aqueous-phase thickeners.

Preferably, the polymers in the cosmetic compositions in accordance with the present invention advantageously have in solution or in dispersion, at 1% active material in water, a viscosity, measured using a Rheomat RM 180 rheometer at 25° C., of greater than 0.1 ps and even more advantageously greater than 0.2 cp, at a shear rate of 200 s⁻¹.

According to one advantageous variant, the composition of the invention comprises one or more associative or nonassociative thickening polymers, particularly A) comprising sugar units in particular derived from the following sugars: glucose; galactose; arabinose; rhamnose; mannose; xylose; fucose; anhydrogalactose; galacturonic acid; glucuronic acid; mannuronic acid; galactose sulfate; anhydrogalactose sulfate and fructose, such as a) gum arabic (branched polymer of galactose, of arabinose, of rhamnose and of glucuronic acid); b) carrageenans and furcellerans (polymers of galactose sulfate and of anhydrogalactose sulfate), c) guar gum (polymer of mannose and of galactose); d) xanthan gum (polymer of glucose, of mannose acetate, of mannose/pyruvic acid and of glucuronic acid); e) gellan gum (polymer of partially acylated glucose, of rhamnose and of glucuronic acid); f) scleroglucan gum (glucose polymer); g) cellulose (glucose polymer); h) starch (glucose polymer); i) nonionic cellulose ethers without a C₁₀-C₃₀ fatty chain; B) polymers resulting from the (co)polymerization of acrylate monomer CH₂═C(R′)—COOR′″ (VIa) and/or of acrylamide monomer CH₂═C(R′)—CO—N(R″)-L-Y⁻ M⁺ (VIb), formulae (VIa) and (VIb) wherein R′ and R″, which may be identical or different, represent a hydrogen atom or a (C₁-C₆)alkyl group such as methyl, preferably hydrogen, R″ represents an alkali metal, an alkaline-earth metal, a hydrogen atom or a (C₁-C₆)alkyl group optionally substituted in particular with one or more hydroxyl, carboxy or amino groups, preferably R′″ represents a hydrogen atom, L representing a cyclic or acyclic, saturated or unsaturated, linear or branched, divalent hydrocarbon-based group, optionally interrupted by one or more heteroatoms such as 0 or N and comprising from 1 to 20 carbon atoms, preferably from 1 to 6 carbon atoms, preferably L represents the divalent group —[C(R′)(R″)]ₚ— with p representing an integer between 1 and 4, preferably 2 and 3 such as 2, R′ and R″ being as defined above, more particularly L represents —C(R′)(R″)—CH₂— or —CH₂—C(R′)(R″)— with R′ and R″ as defined above, preferably R′ and R″ represent a (C₁-C₄)alkyl group such as methyl; Y⁻ represents an anionic group such as carboxylate, phosphate or phosphonate, and M⁺ being a cationic counterion, preferably an alkali metal such as sodium; k) ammonium acrylate homopolymers or copolymers of ammonium acrylate and of acrylamide; C) associative polymers, in particular copolymers comprising, among their monomers, an α,β-monoethylenically unsaturated carboxylic acid and an ester of an α,β-monoethylenically unsaturated carboxylic acid and of an oxyalkylenated fatty alcohol; D) quaternized (poly)hydroxyethylcelluloses modified with groups comprising at least one fatty chain, such as alkyl, arylalkyl or alkylaryl groups comprising at least 8 carbon atoms, or mixtures thereof; alkyl radicals borne by the quaternized celluloses or hydroxyethylcelluloses above preferably comprise from 8 to 30 carbon atoms; E) celluloses or derivatives thereof, modified with groups comprising at least one fatty chain, such as alkyl, arylalkyl or alkylaryl groups or mixtures thereof wherein the alkyl groups are C₈— groups, and in particular nonionic alkylhydroxyethylcelluloses; F) associative guar derivatives such as hydroxypropylguars modified with a fatty chain.

Preferably, the organic thickening polymer(s) iii) is or are present in the composition according to the invention in an amount ranging from 0.01% to 10% by weight relative to the total weight of the composition, more preferentially from 0.1% to 5% by weight relative to the total weight of the composition and even more preferentially from 0.01% to 2.5% by weight relative to the total weight of the composition.

iv) Surfactant

According to one particular embodiment of the invention, the composition also comprises iv) one or more surfactants; they may be anionic, zwitterionic or amphoteric, or nonionic, preferably nonionic or anionic, more preferentially nonionic.

The term “surfactant” is intended to mean a “surface agent”, which is a compound that is capable of modifying the surface tension between two surfaces; surfactants are amphiphilic molecules, i.e. they contain two parts of different polarity, one lipophilic and apolar, and the other hydrophilic and polar.

Among the nonionic surfactants according to the invention, mention may be made, alone or as mixtures, of fatty alcohols, α-diols and alkylphenols, these three types of compound being polyethoxylated, polypropoxylated and/or polyglycerolated and containing a fatty chain comprising, for example, 8 to 22 carbon atoms, the number of ethylene oxide or propylene oxide groups possibly ranging in particular from 2 to 50 and the number of glycerol groups possibly ranging in particular from 2 to 30.

The term “anionic surfactant” is intended to mean a surfactant comprising, as ionic or ionizable groups, only anionic groups. These anionic groups are preferably chosen from the groups —C(O)OH, —C(O)O⁻, —SO₃H, —S(O)₂O⁻, —OS(O)₂OH, —OS(O)₂O⁻, —P(O)OHₑ, —P(O)₂O⁻, —P(O)O₂⁻, —P(OH)₂, ═P(O)OH, —P(OH)O⁻, ═P(O)O⁻, ═POH and ═PO⁻, the anionic parts comprising a cationic counterion such as an alkali metal, an alkaline-earth metal or an ammonium.

As examples of anionic surfactants that may be used in the composition according to the invention, mention may be made of alkyl sulfates, alkyl ether sulfates, alkylamido ether sulfates, alkylaryl polyether sulfates, monoglyceride sulfates, alkylsulfonates, alkylamidesulfonates, alkylarylsulfonates, α-olefin sulfonates, paraffin sulfonates, alkyl sulfosuccinates, alkyl ether sulfosuccinates, alkylamide sulfosuccinates, alkyl sulfoacetates, acylsarcosinates, acylglutamates, alkyl sulfosuccinamates, acylisethionates and N-acyltaurates, polyglycoside-polycarboxylic acid and alkyl monoester salts, acyl lactylates, salts of D-galactoside uronic acids, salts of alkyl ether carboxylic acids, salts of alkylaryl ether carboxylic acids, salts of alkylamido ether carboxylic acids; and the corresponding non-salified forms of all these compounds; the alkyl and acyl groups of all these compounds comprising from 6 to 24 carbon atoms and the aryl group denoting a phenyl group.

The amphoteric or zwitterionic surfactant(s) that can be used in the present invention may especially be derivatives of optionally quaternized secondary or tertiary aliphatic amines containing at least one anionic group, for instance a carboxylate, sulfonate, sulfate, phosphate or phosphonate group, and wherein the aliphatic group or at least one of the aliphatic groups is a linear or branched chain comprising from 8 to 22 carbon atoms.

Mention may be made in particular of (C₈-C₂₀)alkylbetaines, sulfobetaines, (C₈-C₂₀)alkylamido(C₂-C₈)alkylbetaines or (C₈-C₂₀)alkylamido(C₂-C₈)-alkylsulfobetaines.

The amount of surfactants (preferably nonionic or anionic, more preferentially nonionic) preferably ranges from 0.05% to 25% by weight, in particular from 0.1% to 20% by weight, more particularly from 1% to 10% and even more particularly from 2% to 5% by weight relative to the total weight of the composition of the invention.

pH of the Composition

Preferably, the composition according to the invention has a pH of from 5.0 to 6.0. Advantageously, the pH of the composition is between 5.5 and 5.9.

According to one embodiment, the cosmetic composition according to the invention may comprise an acid and a base.

According to one variant, the composition according to the invention can comprise at least one base.

The base is in particular used to increase the pH of the initial aqueous solution. It may also be used to adjust the final pH of the composition to between 5.0 and 6.0 and preferably between 5.5 and 5.9.

The base can be chosen from mineral bases such as, for example, alkali metal hydroxides, sodium hydroxide, potassium hydroxide, ammonium hydroxides, aqueous ammonia, organic bases such as, for example, monoethanolamine, diethanolamine, triethanolamine, triisopropylamine, tris[(2-hydroxy)-1-propyl)]amine, N, N-dimethylethanolamine, 2-amino-2-methyl-1-propanol, 2-amino-2-methyl-1,3-propanediol, triethylamine, dimethylaminopropylamine and amphoteric bases (i.e. bases having both anionic and cationic functional groups) such as primary, secondary, tertiary or cyclic organic amines, amino acids. Examples of amphoteric bases that may be mentioned include glycine, lysine, arginine, taurine, histidine, alanine, valine, cysteine, trihydroxymethylaminomethane (TRISTA), triethanolamine, and any mixture thereof.

According to a particular embodiment, the base of the composition is chosen from sodium hydroxide, potassium hydroxide, ammonium hydroxides, ammonia, monoethanolamine, diethanolamine, triethanolamine, tromethamine, and any mixture thereof. According to one particular embodiment, the base of the composition is chosen from sodium hydroxide, triethanolamine, and a mixture thereof.

According to a particular embodiment, the base of the composition according to the invention is present at a mass concentration of less than 0.5%, or even less than 0.25% by mass, relative to the total mass of the composition.

According to one variant, the composition according to the invention can comprise at least one acid. It may be used to adjust the final pH of the composition to between 5.0 and 6.0 and preferably between 5.5 and 5.9.

The acid may be chosen from mineral acids such as hydrochloric acid, sulfuric acid or nitric acid, organic acids such as acetic acid, lactic acid, glycolic acid, mandelic acid, citric acid or ascorbic acid, and any mixture thereof.

The acid can be chosen from organic acids, such as stearic acid, palmitic acid, myristic acid and any mixture thereof.

According to a particular embodiment, the acid of the composition according to the invention is present at a mass concentration of less than 0.5%, or even less than 0.25% by mass, relative to the total mass of the composition.

Fragrancing Substance

The composition according to the invention comprises at least 5% by weight of at least one fragrancing substance, relative to the total weight of the composition.

The term “fragrancing substance” is intended to mean any fragrance or aroma capable of giving off a pleasant odor.

Fragrances are compositions especially containing the starting materials described in S. Arctander, Perfume and Flavor Chemicals (Montclair, N.J., 1969), in S. Arctander, Perfume and Flavor Materials of Natural Origin (Elizabeth, N.J., 1960) and in Flavor and Fragrance Materials—1991, Allured Publishing Co., Wheaton, Ill.

They may be natural products (essential oils, absolutes, resinoids, resins, concretes) and/or synthetic products (terpene or sesquiterpene hydrocarbons, alcohols, phenols, aldehydes, ketones, ethers, acids, esters, nitriles or peroxides, which may be saturated or unsaturated, and aliphatic or cyclic).

Preferably, the fragrancing substance comprises at least one essential oil.

According to the definition given in international standard ISO 9235 and adopted by the Commission of the European Pharmacopoeia, an essential oil is an odoriferous product generally of complex composition, obtained from a botanically defined plant raw material, either by steam entrainment, or by dry distillation, or via an appropriate mechanical process without heating (cold pressing). The essential oil is usually separated from the aqueous phase via a physical process that does not result in any significant change in the composition.

Methods for Obtaining Essential Oils

The choice of technique depends mainly on the raw material: its original state and its characteristics, its actual nature. The “essential oil/plant raw material” yield may be extremely variable depending on the plants: 15 ppm to more than 20%. This choice determines the characteristics of the essential oil, in particular viscosity, color, solubility, volatility, and richness or poorness in certain constituents.

Steam Entrainment

Steam entrainment corresponds to vaporization, in the presence of steam, of a substance with low water-miscibility. The raw material is placed in contact with boiling water or steam in a still pot. The steam entrains the essential oil vapor, which is condensed in the condenser and recovered as a liquid phase in a Florentine vase (or essence jar), where the essential oil is separated from the water by settling. The aqueous distillate that remains after the steam entrainment, once the separation of the essential oil has been performed, is known as the “aromatic water” or “hydrolate” or “distilled floral water”.

Dry Distillation

The essential oil is obtained by distillation of woods, barks or roots, without addition of water or steam, in a closed chamber designed so that the liquid is recovered at the bottom. Cade oil is the best known example of a product obtained in this way.

Cold Pressing

This production method applies only to citrus fruits (Citrus spp.) via mechanical processes at ambient temperature. The principle of the method is as follows: the zests are torn into pieces and the contents of the secretory sacs that have been broken are recovered by a physical process. The standard process consists in exerting an abrasive action on the entire surface of the fruit under a stream of water. After removal of the solid waste, the essential oil is separated from the aqueous phase by centrifugation. The majority of industrial installations allow simultaneous or sequential recovery of the fruit juices and of the essential oil.

Physicochemical Characteristics

Essential oils are generally volatile and liquid at ambient temperature, which distinguishes them from “set” oils. They are more or less colored and their density is generally less than that of water. They have a high refractive index and most of them deflect polarized light. They are liposoluble and soluble in the usual organic solvents, distillable with steam, and very sparingly soluble in water.

Among the essential oils that may be used according to the invention, mention may be made of those obtained from plants belonging to the following botanical families:

Abietaceae or Pinaceae: conifers

### Amaryllidaceae

### Anacardaceae

Anonaceae: ylang ylang
Apiaceae (for example Umbelliferae): dill, angelica, coriander, sea fennel, carrot, parsley

### Araceae

### Aristolochiaceae

Asteraceae: yarrow, artemisia, camomile, helichrysum

### Betulaceae

### Brassicaceae

Burseraceae: frankincense

### Caryophyllaceae

### Canellaceae

Cesalpiniaceae: copaifera (copaiba balsam)

### Chenopodaceae

Cistaceae: rock rose

### Cyperaceae

### Dipterocarpaceae

Ericaceae: gaultheria (wintergreen)

### Euphorbiaceae

### Fabaceae

Geraniaceae: geranium

### Guttiferae

### Hamamelidaceae

### Hernandiaceae

Hypericaceae: St-John's wort

### Iridaceae

### Juglandaceae

Lamiaceae: thyme, oregano, monarda, savory, basil, marjorams, mints, patchouli,
lavenders, sages, catnip, rosemary, hyssop, balm
Lauraceae: ravensara, sweet bay, rosewood, cinnamon, litsea
Liliaceae: garlic
Magnoliaceae: magnolia

### Malvaceae

### Meliaceae

### Monimiaceae

Moraceae: hemp, hop

### Myricaceae

Myristicaceae: nutmeg
Myrtaceae: eucalyptus, tea tree, paperbark tree, cajuput, backhousia, clove, myrtle

### Oleaceae

Piperaceae: pepper

### Pittosporaceae

Poaceae: lemon balm, lemongrass, vetiver

### Polygonaceae

### Renonculaceae

Rosaceae: roses

### Rubiaceae

Rutaceae: all citrus plants

### Salicaceae

Santalaceae: sandalwood

### Saxifragaceae

### Schisandraceae

Styracaceae: benjoin
Thymelaceae: agar wood

### Tilliaceae

Valerianaceae: valerian, spikenard
Verbenaceae: lantana, verbena

### Violaceae

Zingiberaceae: galangal, turmeric, cardamom, ginger

### Zygophyllaceae.

Mention may also be made of the essential oils extracted from flowers (lily, lavender, rose, jasmine, ylang ylang, neroli), from stems and leaves (patchouli, geranium, petitgrain), from fruit (coriander, aniseed, cumin, juniper), from fruit peel (bergamot, lemon, orange), from roots (angelica, celery, cardamom, iris, sweet flag, ginger), from wood (pinewood, sandalwood, gaiac wood, rose of cedar, camphor), from grasses and gramineae (tarragon, rosemary, basil, lemongrass, sage, thyme), from needles and branches (spruce, fir, pine, dwarf pine) and from resins and balms (galbanum, elemi, benzoin, myrrh, olibanum, opopanax).

Examples of fragrancing substances are especially: geraniol, geranyl acetate, farnesol, borneol, bornyl acetate, linalool, linalyl acetate, linalyl propionate, linalyl butyrate, tetrahydrolinalool, citronellol, citronellyl acetate, citronellyl formate, citronellyl propionate, dihydromyrcenol, dihydromyrcenyl acetate, tetrahydromyrcenol, terpineol, terpinyl acetate, nopol, nopyl acetate, nerol, neryl acetate, 2-phenylethanol, 2-phenylethyl acetate, benzyl alcohol, benzyl acetate, benzyl salicylate, styrallyl acetate, benzyl benzoate, amyl salicylate, dimethylbenzylcarbinol, trichloromethylphenylcarbinyl acetate, p-tert-butylcyclohexyl acetate, isononyl acetate, vetiveryl acetate, vetiverol, a-hexylcinnamaldehyde, 2-methyl-3-(p-tert-butylphenyl)propanal, 2-methyl-3-(p-isopropylphenyl)propanal, 3-(p-tert-butylphenyl)propanal, 2,4-dimethylcyclohex-3-enylcarboxaldehyde, tricyclodecenyl acetate, tricyclodecenyl propionate, 4-(4-hydroxy-4-methylpentyl)-3-cyclohexenecarboxaldehyde, 4-(4-methyl-3-pentenyl)-3-cyclohexenecarboxaldehyde, 4-acetoxy-3-pentyltetrahydropyran, 3-carboxymethyl-2-pentylcyclopentane, 2-n-4-heptylcyclopentanone, 3-methyl-2-pentyl-2-cyclopentenone, menthone, carvone, tagetone, geranylacetone, n-decanal, n-dodecanal, 9-decen-1-ol, phenoxyethyl isobutyrate, phenylacetaldehyde dimethyl acetal, phenylacetaldehyde diethyl acetal, geranonitrile, citronellonitrile, cedryl acetate, 3-isocam phylcyclohexanol, cedryl methyl ether, isolongifolanone, aubepinonitrile, aubepine, heliotropin, coumarin, eugenol, vanillin, diphenyl ether, citral, citronellal, hydroxycitronellal, damascone, ionones, methylionones, isomethylionones, solanone, irones, cis-3-hexenol and esters thereof, musk-indans, musk-tetralins, musk-isochromans, macrocyclic ketones, musk-macrolactones, ethylene brassylate and aliphatic musks, and mixtures thereof.

According to a preferred embodiment of the invention, a mixture of different fragrancing substances that generate in common a note that is pleasant to the user is used.

The fragrancing substances will preferably be chosen such that they produce notes (head, heart and base) in the following families:

citrus,
aromatic,
floral note, in particular pink flowers and white flowers,
spicy,
woody,
gourmand,
chypre,
fougere,
leathery,
musk.

The fragrancing compositions of the invention preferably contain from 5% to 30% by weight of fragrancing substance, better still from 10% to 25% by weight and in particular from 15% to 25% by weight relative to the total weight of the composition.

According to a particular embodiment, the fragrancing composition according to the invention also comprises cosmetically acceptable active agents and/or excipients.

The term “cosmetically acceptable” is intended to mean compatible with the skin and/or its integuments, having a pleasant color, odor and feel and not causing any unacceptable discomfort (stinging, tautness or redness) liable to discourage the consumer from using this composition.

Preferably, the fragrancing composition according to the invention comprises less than 2% by weight of oil relative to the total weight of the composition, preferably less than 1% by weight of oil, preferably less than 0.5% by weight of oil. Preferably, the fragrancing composition according to the invention is free of oil. The oil is in this case distinct from the fragrancing substance. The term “oil” is intended to mean a nonaqueous fatty compound that is liquid at ambient temperature (25° C.) and atmospheric pressure (760 mmHg). In particular, the oil is in this case distinct from essential oils.

Preferably, the fragrancing composition according to the invention is constituted of: a physiologically acceptable aqueous medium comprising at least ethanol; at least one sulfonic polymer chosen from homopolymers of acrylamido-2-methylpropanesulfonic acid or salts thereof and copolymers of acrylamido-2-methylpropanesulfonic acid or salts thereof and of one or more nonionic monomers; at least one crosslinked copolymer of acrylic acid and of a C₁₀-C₃₀ alkyl acrylate; and at least 5% by weight of at least one fragrancing substance, relative to the total weight of the composition.

The invention also relates to a method of cosmetic and/or esthetic care of, in particular for fragrancing, keratin materials, comprising the topical application to keratin materials, preferably the skin, of a composition according to the invention.

Concrete but in no way limiting examples illustrating the invention will now be given.

In the examples, the temperature is ambient temperature (20° C.) and is expressed in degrees Celsius, unless otherwise indicated, and the pressure is atmospheric pressure, unless otherwise indicated.

In the examples, the amounts of the composition ingredients are given as weight percentages relative to the total weight of the composition.

### EXAMPLE 1

### Fragrancing Composition According to the Invention and Comparative Compositions

I/ The composition according to the invention according to table 1 is prepared according to the following process:
Water, alcohol (ethanol) and polyols are mixed at ambient temperature with stirring in a Moritz mixer;
The Carbopol Ultrez 21 Polymer is added and the mixture is stirred;
The fragrance is added;
Finally, Sepigel 305 and then the base are added.

TABLE 1

| | Amount (% by weight |
|---|---|
| | relative to the total |
| | weight of composition) |
| | Composition according |
| Ingredients | to the invention |
| 2-Amino-2-methyl-1-propanolamino- | 0.06 |
| methylpropanol | |
| Fragrance | 20 |
| Acrylamide/sodium acrylamido-2- | 0.5 |
| methylpropanesulfonate copolymer | |
| as an inverse emulsion at 40% in | |
| isoparaffin/water (Sepigel 305 from | |
| SEPPIC) | |
| Crosslinked acrylic acid/C₁₀-C₃₀ alkyl | 0.4 |
| methacrylate copolymer | |
| (Carbopol Ultrez 21 Polymer from | |
| Lubrizol) | |
| Glycerol | 2 |
| Ethanol | 21 |
| Dipropylene glycol | 15 |
| Water | qs 100 |

This composition according to the invention has a pH of 5.67 and a viscosity of 28 poises (2.8 Pa·s) measured according to the protocol described above. When the composition is stored at 4° C. for one month, it has a pH of 5.5 and a viscosity of 26 poises (2.6 Pa·s) measured according to the protocol described above.

This composition comprises 20% by weight of fragrance concentrate, in an aqueous-alcoholic gel. It also contains 21% by weight of ethanol. It is cosmetically advantageous, since it has a powdery finish, has a pleasant and easy uptake, and is very fresh and pleasant on application. The composition is stable and homogeneous even after storage for one month at a wide temperature range: 4° C. or at 45° C.

II/ The comparative compositions A to D according to table 2 are prepared according to the same process as described for the composition according to the invention, but without Sepigel 305 and/or without Carbopol Ultrez 21. For comparative composition C, Aristoflex SNC is introduced in the same way as Sepigel 305.

TABLE 2

| | Amount (% by weight | Amount (% by weight | Amount (% by weight | Amount (% by weight |
|---|---|---|---|---|
| | relative to the total | relative to the total | relative to the total | relative to the total |
| | weight of composition) | weight of composition) | weight of composition) | weight of composition) |
| Ingredients | Formula A | Formula B | Formula C | Formula D |
| 2-Amino-2-methyl-1- | 0.06 | 0.06 | 0.06 | 0.06 |
| propanolaminomethyl- | | | | |
| propanol | | | | |
| Fragrance | 20 | 20 | 20 | 20 |
| AMPS/ethoxylated C₁₆/C₁₈ | — | — | 0.5 | — |
| alkyl dimethacrylate (8 mol | | | | |
| of EO) 80/20 copolymer at | | | | |
| 92% in a water/butanol | | | | |
| mixture (5/3) | | | | |
| (Aristoflex SNC from | | | | |
| Clariant) | | | | |
| Acrylamide/sodium | — | 0.5 | — | 0.9 |
| acrylamido2- | | | | |
| methylpropanesulfonate | | | | |
| copolymer as an inverse | | | | |
| emulsion at 40% in | | | | |
| isoparaffin/water | | | | |
| (Sepigel 305 from | | | | |
| SEPPIC) | | | | |
| Crosslinked acrylic | 0.4 | — | — | — |
| acid/C₁₀-C₃₀ alkyl | | | | |
| methacrylate copolymer | | | | |
| (Carbopol Ultrez 21 | | | | |
| Polymer from Lubrizol) | | | | |
| Glycerol | 2 | 2 | 2 | 2 |
| Ethanol | 21 | 21 | 21 | 21 |
| Dipropylene glycol | 15 | 15 | 15 | 15 |
| Water | qs 100 | qs 100 | qs 100 | qs 100 |

The comparative formula A has a pH of 6 and a viscosity of 20 poises (2 Pa·s) measured according to the protocol described above. It is thick, it has a more aqueous texture and is not as easy to take up as the formula according to the invention.

The comparative formula B has a pH of 5.6 and a viscosity of 12 poises (1.2 Pa·s) measured according to the protocol described above. It is very fluid, has a more sticky texture and is less fresh than the formula according to the invention.

The comparative formula C breaks from TO. It is therefore unstable.

As for the comparative formula D, it is much too fluid and difficult to take up. It has a pH of 5.5 and has a viscosity of 19 poises (1.9 Pa·s) measured according to the protocol described above. When the composition is stored at 4° C. for one month, it has a pH of 5.2 and a viscosity of 4.9 poises (0.49 Pa·s) measured according to the protocol described above. When the composition is stored at 45° C. for one month, it has a pH of 5.2 and a viscosity of 5.7 poises (0.57 Pa·s) measured according to the protocol described above.

Thus, these results show that only the fragrancing composition according to the invention, which contains a copolymer of acrylamido-2-methylpropanesulfonic acid or salts thereof and of one or more nonionic monomers and a crosslinked copolymer of acrylic acid and of C₁₀-C₃₀ alkyl acrylate, is stable and cosmetically advantageous.

III/ A second composition according to the invention according to table 3 is prepared according to the process described above (see section I). This second composition according to the invention this time comprises an acrylamido-2-methylpropanesulfonic acid homopolymer.

TABLE 3

| | Amount (% by weight |
|---|---|
| | relative to the total |
| | weight of composition) |
| | Composition according |
| Ingredients | to the invention |
| 2-Amino-2-methyl-1-propanolamino- | 0.06 |
| methylpropanol | |
| Fragrance | 20 |
| Polyacrylamidomethylpropanesulfonic | 0.5 |
| acid partially neutralized with aqueous | |
| ammonia and highly crosslinked | |
| (Hostacerin AMPS ® from Clariant) | |
| Crosslinked acrylic acid/C₁₀-C₃₀ alkyl | 0.4 |
| methacrylate copolymer | |
| (Carbopol Ultrez 21 Polymer from | |
| Lubrizol) | |
| Glycerol | 2 |
| Ethanol | 21 |
| Dipropylene glycol | 15 |
| Water | qs 100 |

This composition according to the invention has a pH of 5.4 and a viscosity of 52 poises (5.2 Pa·s) measured according to the protocol described above (with spindle 4). The composition exhibits good stability.

1. A fragrancing composition, comprising:
a physiologically acceptable aqueous medium comprising at least ethanol;
i) one or more sulfonic polymers chosen from homopolymers of acrylamido-2-methylpropanesulfonic acid or salts thereof and copolymers of acrylamido-2-methylpropanesulfonic acid or salts thereof and of one or more nonionic monomers;
ii) one or more crosslinked copolymers of acrylic acid and of C₁₀-C₃₀ alkyl acrylate; and
iii) optionally one or more organic thickening polymers other than i) and ii);
at least 5% by weight of at least one fragrancing substance, relative to the total weight of the composition.

2. The fragrancing composition as claimed in claim 1, wherein the aqueous medium comprises at least one other organic solvent which is soluble in water at 25° C., chosen from linear or branched C₃-C₄ alkanols; polyols having from 2 to 20 carbon atoms; and mixtures thereof.

3. The fragrancing composition as claimed in claim 1, which comprises from 15 to 40% by weight of ethanol, relative to the total weight of the composition.

4. The fragrancing composition as claimed in claim 1, which comprises i) one or more homopolymers of acrylamido-2-methylpropanesulfonic acid or salts thereof, said homopolymer(s) comprising, distributed randomly:
from 90% to 99.9% by weight of units of general formula (1) below: 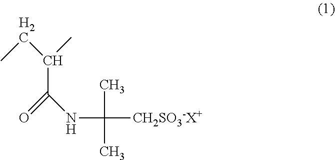
wherein X⁺ denotes a proton, a cation of an alkali metal, a cation of an alkaline-earth metal, or the ammonium ion, it being possible for at most 10 mol % of the cations X⁺ to be protons H⁺;
and from 0.01% 10% by weight of crosslinking units originating from at least one monomer having at least two olefin double bonds;
the proportions by weight being defined with respect to the total weight of the polymer.

5. The fragrancing composition as claimed in claim 1, which comprises i) one or more copolymers of acrylamido-2-methylpropanesulfonic acid or salts thereof and of one or more nonionic monomers, which are chosen from:
copolymers of acrylamido-2-methylpropanesulfonic acid and of vinylpyrrolidone,
crosslinked acrylamide/sodium acrylamido-2-methylpropanesulfonate copolymers, and
copolymers of acrylamido-2-methylpropanesulfonic acid and of hydroxyethyl acrylate.

6. The fragrancing composition as claimed in claim 1, wherein the concentration of acrylamido-2-methylpropanesulfonic acid homopolymer or copolymer in terms of active material ranges from 0.05% to 1% by weight relative to the weight total of the composition.

7. The fragrancing composition as claimed in claim 1, wherein the concentration of crosslinked copolymer of acrylic acid and of C₁₀-C₃₀ alkyl acrylate ii) in terms of active material ranges from 0.05% to 1% by weight relative to the total weight of the composition.

8. The fragrancing composition as claimed in claim 1, which comprises from 5% to 30% by weight of fragrancing substance relative to the total weight of the composition.

9. The fragrancing composition as claimed in claim 1, which has a viscosity of between 9 and 40 poises.

10. The fragrancing composition as claimed in claim 1, which comprises less than 2% by weight of oil relative to the total weight of the composition.

11. The fragrancing composition as claimed in claim 1, which comprises one or more organic thickening polymers other than i) and ii), which may be associative or nonassociative.

12. The fragrancing composition according to claim 1, which comprises one or more thickening polymers in an amount ranging from 0.01% to 10% by weight relative to the total weight of the composition.

13. The fragrancing composition as claimed in claim 1, which comprises one or more surfactants.

14. The fragrancing composition as claimed in claim 1, which is constituted of:
the physiologically acceptable aqueous medium comprising at least ethanol;
i) the one or more sulfonic polymers chosen from homopolymers of acrylamido-2-methylpropanesulfonic acid or salts thereof and copolymers of acrylamido-2-methylpropanesulfonic acid or salts thereof and of one or more nonionic monomers;
ii) the one or more crosslinked copolymers of acrylic acid and of C₁₀-C₃₀ alkyl acrylate; and
iii) optionally the one or more organic thickening polymers other than i) and ii);
at least 5% by weight of the at least one fragrancing substance, relative to the total weight of the composition.

15. A method of cosmetic and/or esthetic care comprising the topical application to keratin materials of a composition as claimed in claim 1.

16. The fragrancing composition as claimed in claim 2, wherein the linear or branched C₃-C₄ alkanol is isopropanol, propanol or butanol and the polyols having from 2 to 6 carbon atoms.

17. The fragrancing composition as claimed in claim 2, which comprises from 15 to 30% by weight of ethanol, relative to the total weight of the composition.

18. The fragrancing composition as claimed in claim 2, which comprises i) one or more homopolymers of acrylamido-2-methylpropanesulfonic acid or salts thereof, said homopolymer(s) comprising, distributed randomly:
from 90% to 99.9% by weight of units of general formula (1) below: 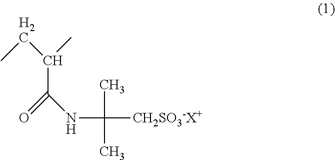
wherein X⁺ denotes a proton, a cation of an alkali metal, a cation of an alkaline-earth metal, or the ammonium ion, it being possible for at most 10 mol % of the cations X⁺ to be protons H⁺;
and from 0.01% 10% by weight of crosslinking units originating from at least one monomer having at least two olefin double bonds;
the proportions by weight being defined with respect to the total weight of the polymer.

19. The fragrancing composition as claimed in claim 3, which comprises i) one or more homopolymers of acrylamido-2-methylpropanesulfonic acid or salts thereof, said homopolymer(s) comprising, distributed randomly:
from 90% to 99.9% by weight of units of general formula (1) below: 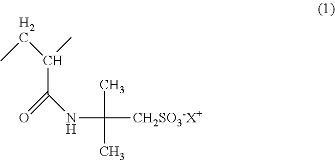
wherein X⁺ denotes a proton, a cation of an alkali metal, a cation of an alkaline-earth metal, or the ammonium ion, it being possible for at most 10 mol % of the cations X⁺ to be protons H⁺;
and from 0.01% 10% by weight of crosslinking units originating from at least one monomer having at least two olefin double bonds;
the proportions by weight being defined with respect to the total weight of the polymer.

20. The fragrancing composition as claimed in claim 2, which comprises i) one or more copolymers of acrylamido-2-methylpropanesulfonic acid or salts thereof and of one or more nonionic monomers, which are chosen from:
copolymers of acrylamido-2-methylpropanesulfonic acid and of vinylpyrrolidone,
crosslinked acrylamide/sodium acrylamido-2-methylpropanesulfonate copolymers, and
copolymers of acrylamido-2-methylpropanesulfonic acid and of hydroxyethyl acrylate.
